SEQUENCE LISTING (1 sequence)

SEQ ID NO: 1 - DNA, 3410 bp, Tetrahymena thermophila
aagcttttttatttagtaaaaatttatctttcatttaatttataatcatatttatatctttgtatttaaaaacaataaataatagcaatgactatactatccttgactcccattaatcatttgtgataataaaataagaaaatggaaataattgagatataaaatcatatagcaacgctcttgattctttgtagtgtttaaaaatagtttgcctacttatgcatagtatacctttgacatatattcctgtatttgcatgaacaaacatatctaaaaacaaaattaactaacaaataagaataatatctaatctaaatagttaatgaagctgagcaccaaagctaaagcaaacaaaatagcaaaaaaagcatataagagatgcaaaaatttttaaagcctaccaaacttctatgcatcttgtatgtggtaaaaaaataggtatttatttatagaaatattctaaacaattaattttctacttgctctttttaactgctttgtcaaaataaatcatagtattatacaatttacttttttcagatattattttttaactgaacattttattagcttaaaaatattgatgcttttcatccttaaaatgagatctgtcatttatcatttctgtttacctccatgttattagtctttgcattctacttcgagcaaactataaaaatatttgagcatactttctaaatctaattttaaaagatatttcttaaagtctttaaagttagttttttttattgaagaataattagctttaatcaagtaaaatgttttattgttaatttatttaattagatttattttataagttattttggctatggttagcaaattatatctttaaatcctttttcatacttattttgtatattcaaaatctcaacaaaagctaatttctactttttaattaattaatttctactaatttagtaaaaaataaaaatttgcaaattttttaattaattaaaaatcctttgtaacgttttgatgtaattttaagagtccttggtcttaacactaaaaacttatttaaagaaaaataaatcaataatgcaaatttattttattcaaggcttgcttttaaagcattaaataggattaaacaatataaattttagctatttaaattaaatttattatttttaatgattaaaattattaaacttgaataaatgatcctaattctttttgttatttatattggaataacatttaagtaaatcaacataaaaatgactttaatacaatttactattcttcatagtagttgcttgagcttttgaagtatgaattaggtttaaatatcatattattttagctgtttaaattaaatttattatttttgatgattaaaataattcagattttatgaatgatactacttattttaaatttcatgtaaaaataatattagtgatggtattaaaaatataattagattttttaaaaattgtttggaaataagtacttaattccaaatttcttttaatttttaagaattaaattagtttatttttatatattaattcactaaataaaaagataagtaatatatttagtgcacaatgtttgaatgtttttttgataaatttgtaaattaaatctatttaagtcagtagaattttagatattttcatagattttttaataagtagacaatttatttctaaaaaatatttaaaaataaaaaataataagggttttgaataactcctttaatttaaatacacatttttaaattttttttagctctttaaatattcataaaaataaaaaataactaactaaaaataaataaaaagataataatgattaaaggtataatactgaataagaaaaaacataatagagtacttattttttatatcactatttttaatatcttgaaagcaaaacttttttatatatcttaaaatatattgtatcgtttattcaattattttctttaaatttcaaatatattgataaaaaagatgacatgttttttaaagaaaacatgaaatataaaatagataaatatcaattattttatttattaaatatataagctgctcaaaacatagctcattcatcaattataatatgtgaatcattaattttcaaaatattactcattatttaggctatcatttattttttattttcaattatccgtttctattatattttaatattaagttgtgattcttgaattttgtgtcatgaattatttgtaaatctttttatttctgataaaaaatataaattgattgactcatgatttaaatcatgagtcaacctaactaattttcaaaattcttctattctaaaatatagatgtgattcttgaatctctcttgaatataaagtaattttttatatttctgatataattcttagctacgtgattcacgatttatgcaatgatccatataaaataatgtaaatagtgtatatatatatattcgtcttttttattctttatataatttaaaaaaattaaaaaaatttaataaagctctaataaaataaataataatactaaacttaaaataatggataaagttaatagctgttgctgtggtgtaaatgctaagccatgctgcacagaccctaacagcggatgttgctgcgtaagtaagactgataattgctgcaaatctgacacaaaggaatgttgcactggtacaggagagggatgcaaatgtgttaattgcaagtgctgcaaacctcaagcaaattgttgctgtggagttaatgctaaaccctgctgctttgatcccaatagtggatgttgctgtgttagtaaaactaataactgttgtaaatctgatactaaagaatgctgtactggtactggagaaggatgcaaatgtacaagttgctaatgctgcaaacctgtttaataaggatgttgttgtggtgataaagctaaggcttgctgtactgatccaaacagtggatgttgctgctcaaataaggcaaataaatgctgtgatgctacttcaaagcaagagtgttagacttgttaatgttgtaaatgaaataaatatgttaattaaaatttaaaatatgttgatgttgtaattcataattgcataaaatcagtttcgtattttattaaaaactttaatattatcatcctaaaatttattaaaaaaaactgttattaataaatcaataattatcgctatttcatagatttttttaaatgcaaaagcaagtatgttatttaaattaagtaaacattgattgaaagtattatctacagaaaaaaaaaatatataatttactttttaatcaatttaattggaaattattttatattttttaaaaatgaaaaaacatatttttataataaacattcaaatttattttacaaaatttatataaaaatctttaataattcaattaattga

(19) United States
(12) Patent Application Publication
(10) Pub. No.: US 2003/0027192 A1
(43) Pub. Date: Feb. 6, 2003

(54) Tetrahymena metallothionein gene promoter and its use

(76) Inventors: Martin A. Gorovsky, Fairport, NY (US); Yuhua Shang, Rochester, NY (US); Xiaoyuan Song, Rochester, NY (US)

Correspondence Address: Michael L. Goldman, NIXON PEABODY LLP, Clinton Square, P.O. Box 31051, Rochester, NY, 14603-1051, US

(21) Appl. No.: 10/196,063

(22) Filed: Jul. 15, 2002

Related U.S. Application Data
(60) Provisional application No. 60/305,167, filed on Jul. 13, 2001.
(60) Provisional application No. 60/317,322, filed on Sep. 5, 2001.

(51) Int. Cl. C12Q 1/68; C07H 21/04; C12N 9/02; C12N 15/74; C12P 21/02; C12N 1/21

(52) U.S. Cl. 435/6; 435/69.1; 435/189; 435/320.1; 435/252.3; 536/23.2

(57) ABSTRACT

An isolated DNA molecule which includes a promoter-effective region of a Tetrahymena metallothionein gene, as well as a chimeric gene, expression vectors, and host cells containing the same. Also disclosed is a method of expressing an RNA or a polypeptide of interest using a chimeric gene of the invention.

This application claims the benefit of U.S. Provisional Patent Application Serial No. 60/305,167 filed Jul. 13, 2001 and U.S. Provisional Patent Application Serial No. 60/317,322 filed Sep. 5, 2001, each of which is hereby incorporated by reference in its entirety.

The work underlying this application was supported at least in part by National Institutes of Health Grant No. GM 26973. The U.S. government may have certain rights in this invention.

### FIELD OF THE INVENTION

The present invention relates generally to recombinant molecular biology and more specifically to a promoter and its use in recombinant molecular biology.

### BACKGROUND OF THE INVENTION

Tetrahymena thermophila is a ciliated protozoan that grows rapidly to high densities. Its relatively large size, nuclear dimorphism, and well developed techniques for genetic analyses, cytological studies, and cell fractionation make it a useful eukaryotic model to study diverse molecular, cellular, and developmental processes (see Asai & Forney, Methods in Cell Biology: Tetrahymena thermophila (Academic Press, San Diego) (2000)). Several fundamental and evolutionarily conserved phenomena were first identified in T. thermophila, including the discovery of dynein (Gibbons, Proc. Natl. Acad. Sci. USA 50:1002-1010 (1963)), the structure of telomeres (Blackburn & Gall, J. Mol. Biol. 120:33-53 (1978)), the identification of telomerase as a ribonucleoprotein enzyme (Greider & Blackburn, Cell 51:887-898 (1987)), self-splicing RNA (Zaug & Cech, Science 231:470-475 (1986)), and the relationship between transcription factors and histone modification (Brownell et al., Cell 84:843-851 (1996)).

In recent years, experimental analyses in Tetrahymena have been augmented by the development of methods for DNA-mediated transformation of the somatic macronucleus, first by microinjection of individual cells (Tondravi & Yao, Proc. Natl. Acad. Sci. USA 83:4369-4373 (1986)), then en masse by electroporation (Gaertig & Gorovsky, Proc. Natl. Acad. Sci. USA 89:9196-9200 (1992)) or biolistically (Cassidy-Hanley et al., Genetics 146:135-147 (1997)), and by the discovery that transformation in Tetrahymena occurs largely, if not entirely by homologous integration (Yao & Yao, Proc. Natl. Acad. Sci. USA 88:9493-9497 (1991)). Coupled with the introduction of heterologous drug-resistance gene markers (Kahn et al., Proc. Natl. Acad. Sci. USA 90:9295-9299 (1993); Xia et al., J. Cell Biol. 149:1097-1106 (2000)), these methods enabled facile gene replacement, allowing gene disruption (Gaertig & Kapler, Methods Cell Biol. 62:485-500 (2000)) and insertion of mutant genes with flanking selectable markers (Yu & Gorovsky, Methods Cell Biol. 62:549-559 (2000)). They also led to techniques for transformation of the germ-line micronucleus which, again, occurs by homologous integration (Bruns & Cassidy-Hanley, Methods Cell Biol. 62:501-512 (2000)). Germ-line transformation led, in turn, to the creation of germ-line knockout heterokaryon strains (Hai et al., Methods Cell Biol. 62:513-531 (2000)), in which both copies of an essential gene are disrupted in the transcriptionally silent micronucleus whereas the transcriptionally active, somatic macronucleus contains wild-type genes. When two strains that are knockout heterokaryons for the same essential gene are mated, their progeny die unless they are transformed with a version of the gene that supports growth. Although these methods have enabled many studies (Xia et al., J. Cell Biol. 149:1097-1106 (2000); Dou & Gorovsky, Mol. Cell 6:225-231 (2000); Chilcoat et al., Proc. Natl. Acad. Sci. USA 98:8709-8713 (2001); Ren & Gorovsky, Mol. Cell 7:1329-1335 (2001)), molecular genetic analyses in Tetrahymena were still limited by relatively low efficiencies of transformation, precluding cloning genes by complementation of function or suppression of mutations, and by the absence of a regulatable expression system. It would be useful, therefore, to identify an inducible-repressible promoter in Tetrahymena.

The present invention is directed to overcoming these and other deficiencies in the art.

### SUMMARY OF THE INVENTION

A first aspect of the present invention relates to an isolated DNA molecule comprising a promoter-effective region of a Tetrahymena metallothionein gene.

A second aspect of the present invention relates to a chimeric gene that includes: a first DNA encoding an mRNA molecule or a protein or polypeptide; a second DNA molecule including the promoter-effective region of a Tetrahymena metallothionein gene of the present invention operably linked 5′ to the first DNA molecule; and a third DNA molecule comprising a 3′ regulatory region operably linked 3′ to the first DNA molecule. Expression vectors, host cells, and transgenic Tetrahymena organisms containing the chimeric gene are also disclosed.

A third aspect of the present invention relates to a method of expressing an RNA or a polypeptide of interest, the method including: providing a chimeric gene of the present invention and transforming a host cell with the chimeric gene under conditions effective to express the RNA or polypeptide in the host cell.

A fourth aspect of the present invention relates to an empty expression vector that includes: a first DNA molecule including one or more restriction enzyme cleavage sites; a second DNA molecule including the promoter-effective region of a Tetrahymena metallothionein gene of the present invention coupled 5′ of the first DNA molecule; and a third DNA molecule including a 3′ regulatory region operably linked 3′ of the first DNA molecule; wherein insertion of a DNA molecule into the first DNA molecule at a cleavage site operably couples the third DNA molecule to the second and third DNA molecules.

Metallothioneins (MTTs) are highly conserved, low molecular weight, cysteine-rich metal-binding proteins whose primary function is unknown, but who are generally considered to play a role in the homeostasis of metals such as zinc and copper and in the detoxification of cadmium (Miles et al. Crit. Rev. Biochem. Mol. Biol. 35:35-70 (2000)). The synthesis of many metallothioneins, including those of Tetrahymena pyriformis and T. thermophila, can be induced by heavy metals, such as zinc, copper, and cadmium (Piccinni et al., Eur. J. Protistol. 26:176-181 (1990)). Metal-responsive metallothionein promoters have been used successfully to regulate gene expression in other systems (Palmiter et al., Science 222:809-814 (1983); Karin et al., Cell 36:371-339 (1984)). The present invention relates to the cloning and regulation of MTT1, a gene encoding a Cd²⁺-inducible metallothionein T. thermophila. This promoter can greatly increase the efficiency of many aspects of DNA-mediated transformation in Tetrahymena, including somatic and germ-line gene disruption and rescue of knockout heterokaryons. The MTT1 promoter also can be used to overexpress homologous or heterologous genes and to create a conditional lethal mutation of an essential gene.

### BRIEF DESCRIPTION OF THE DRAWINGS

FIG. 1 shows the nucleotide sequence (SEQ ID No: 1) of the Tetrahymena thermophila metallothionein gene (MTT1). The coding sequence extends from nt 2547-3035 (489 nt, shown in bold typeface), with the 5′ flanking region extending from nt 1-2546 and the 3′ flanking region extending from nt 3036 to 3410 (375 nt).

FIGS. 2A-C illustrate Northern blot analyses of MTT1 induction. FIG. 2A shows that transcription of the MTT1, but not the GTU1 gene is induced by cadmium in growing cells. Total RNA was analyzed from wild-type CU428 cells grown overnight in SPP medium containing the indicated concentrations of CdCl₂. FIG. 2B shows that transcription of the MTT1 gene is induced by cadmium in starved and mating cells. To starve cells, a culture of log phase CU428 cells was washed twice and then incubated at 30° C. without shaking in 10 mM Tris with the indicated concentration of CdCl₂. To obtain mating cells, CU428 and B2086 cells were starved overnight as described above, mixed to initiate mating, and incubated at 30° C. without shaking in 10 mM Tris with the indicated concentration of CdCl₂. Total RNA was isolated either 2 or 24 h after starvation (starved cells) or mixing (mating cells). FIG. 2C shows that induction and repression of the MTT1 promoter occur rapidly. In +CdCl₂, RNA was analyzed from log-phase wild-type CU428 cells incubated in SPP containing 1.0 μg/ml CdCl₂ for the indicated times. In −CdCl₂, wild-type CU428 cells were grown in SPP with 1.0 μg/ml CdCl₂ overnight and then washed twice with SPP medium. Total RNA was isolated at the indicated times. All Northern blots were probed with a MTT1 or GTU1 coding sequence probe or with a 25S rRNA probe as a control for loading.

FIGS. 3A-B illustrate the transformation of Tetrahymena with a neomycin resistance gene driven by the MTT1 promoter. FIG. 3A shows the structure of the endogenous MTT1 gene and the insert in plasmid pTTMN which contains the neo1 gene flanked by MTT1 5′ and 3′ noncoding sequences. FIG. 3B shows the effect of cadmium concentration on biolistic transformation of Tetrahymena by pTTMN. Wild-type CU428 cells were transformed (see Materials and Methods) and plated at the concentrations of CdCl₂ indicated on the abscissa. The percentage of paromomycin-resistant transformants per 96-well plate was plotted relative to the number obtained with 2.0 μg/ml CdCl₂.

FIGS. 4A-C illustrate that the MTT1 promoter improves the efficiency of DNA-mediated, biolistic transformation of Tetrahymena. FIG. 4A shows that the MTT1 promoter-driven neo3 cassette gives higher somatic transformation rates than the HHF1 promoter-driven neo2 cassette. Four different GTU1 knockout constructs are shown. All cassettes use the same BTU2 3′-flanking region, but they differ in the 5′-flanking region. In p₆₆GMN, neo3 expression is driven by 2.5 kb of MTT1 5′-flanking region, and in p_{Δ}GN, neo2 is driven by the HHF1 promoter. In p_{Δ}GMMII, neo3 is driven by 900 bp of MTT1 5′-flanking sequence. In p_{Δ}GMM, neo3 is driven by 600 bp of MTT1 5′-flanking sequence. Three micrograms of DNA were used in each transformation. After transformation, the CU428 cells were refed in 1X SPP with 1.0 μg/ml CdCl₂ for 3 to 6 h before adding 120 μg/ml paromomycin followed by plating. The number of transformants was obtained by counting the number of wells with viable transformants in 96-well plates at known dilutions. FIG. 4B shows that the MTT1 promoter-driven neo3 cassette enables both somatic and germ-line knockout of the ngoA gene where the HHF1-driven neo2 cassette fails. Two different ngoA knockout constructs are shown. Both contain the same ngoA-flanking sequences. p_{Δ}NgoAMT contains the neo3 cassette driven by the MTT1 promoter, and p_{Δ}NgoAH4 contains neo2 driven by the HHF1 promoter. Wild-type CU428 cells were mated with B2086 cells, and 3 μg DNA was used in each transformation. After transformation, cells were starved in 10 mM Tris overnight and then refed in SPP containing 1.2 μg/ml CdCl₂ for 3 to 6 h before addition of 80 μg/ml paromomycin and plating. After 3 days, more than 700 transformants were obtained. Paromomycin-resistant transformants were tested for sensitivity to 6-methylpurine in SPP. The Pm/6-methylpurine double-resistant transformants were further tested by Southern blotting to determine whether the neo cassettes were in the correct locus and for ability to sexually transmit the knockout phenotype. Two neo3 transformants were actual germ-line knockout transformants. FIG. 4C shows that the MTT1 promoter increases the rescue efficiency of knockout heterokaryons. Two constructs were used to rescue GTU1 germ-line knockout heterokaryons. Mating cells were transformed with 3 μg DNA. Transformants were selected in 60 μg/ml paromomycin for 4 days and the number of transformants calculated by counting the number of wells with viable transformants in 96-well plates at known dilutions.

FIGS. 5A-C illustrate that an essential gene regulated by the MTT1 promoter behaves as a conditional mutation. In FIG. 5A, wild-type Cu428 (●) or cTTMG (⋄) cells, in which the GTU1-coding sequence was regulated by the MTT1 promoter, were resuspended in SPP medium without cadmium. Cells were counted at various times after suspension. Growth of the cTTMG cells without cadmium slowed at about 11 h relative to wild-type cells. Growth of cTTMG cells in the presence of cadmium is indistinguishable from that of wild-type cells. FIGS. 5B-C show that the shape and microtubule distribution of cTTMG cells was disrupted after depletion of cadmium. Wild-type cells (FIG. 5B) or cells containing the MTT1-GTU1 chimeric gene (FIG. 5C) were grown in normal SPP media for 24 h and then were fixed and stained with anti-α-tubulin antibody. The shape and microtubule distribution of cTTMG cells grown in the presence of cadmium is indistinguishable from that of wild-type cells.

FIGS. 6A-B illustrate that the MTT1 promoter allows overexpression of a foreign gene in Tetrahymena. FIG. 6A shows schematic maps of the target taxol-sensitive BTU1-K350M locus of T. thermophila and two transforming plasmid inserts in which the BTU1-coding region was replaced by IAG48[G1] sequences encoding a surface antigen from the fish parasite, Ich. The expression of the IAG48[G1] gene is driven by either the BTU1 (pBICH3) or the MTT1 (pMTT-BICH3) promoter. FIG. 6B shows the results of Western blot with an anti-Ich surface antigen antibody. In the presence of cadmium, expression of the IAG48[G1] gene driven by the MTT1 promoter is much higher than that driven by the BTU1 promoter.

### DETAILED DESCRIPTION OF THE INVENTION

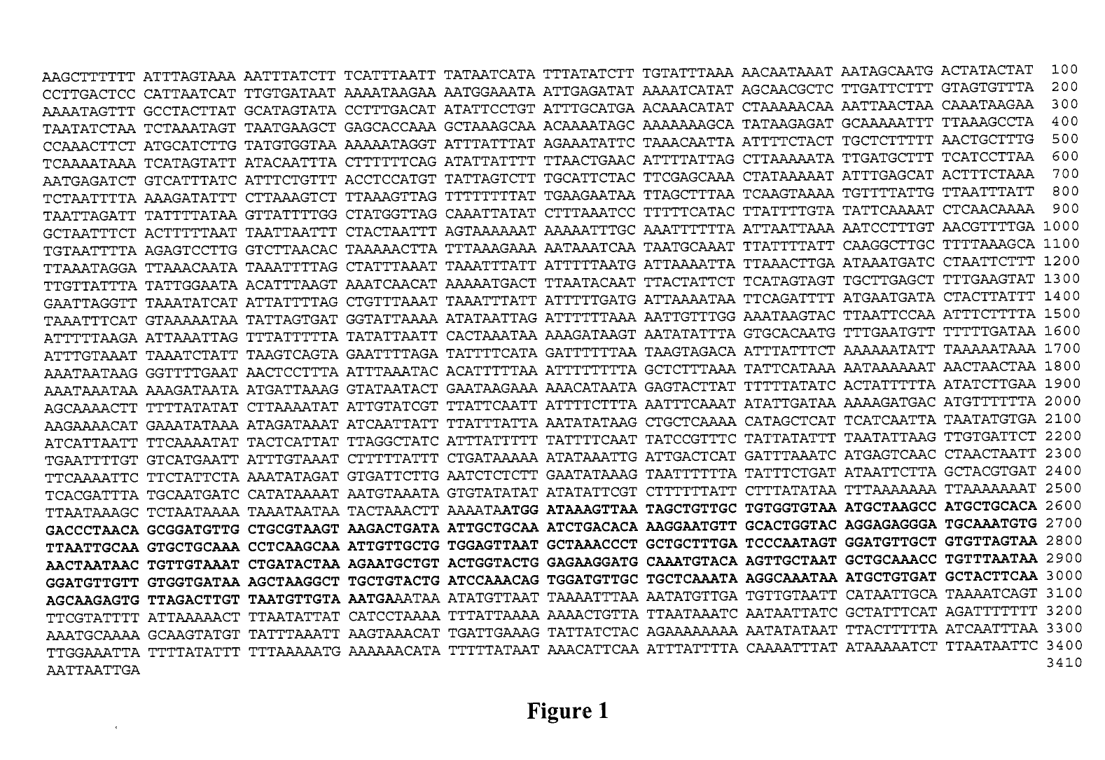

One aspect of the present invention relates to an isolated DNA molecule that includes a promoter-effective region of a Tetrahymena metallothionein gene. The entire sequence of the Tetrahymena thermophila metallothionein gene (MTT1) is illustrated in FIG. 1. It is believed that the promoter effective region of this gene is the first isolated inducible-repressible promoter of Tetrahymena.

The promoter-effective region preferably includes greater than about 600 bp upstream (5′) of the Tetrahymena metallothionein gene start codon (nt 2547-2549). Alternatively, the promoter-effective region includes at least about 900 bp upstream of the start codon, at least about 1.6 kb upstream of the start codon, or at least about 2.5 kb upstream of the Tetrahymena metallothionein gene start codon. Of these suitable promoter-effective regions, the region that includes at least about 900 bp upstream of the start codon is most preferred.

Other fragments of the nucleotide sequence given as SEQ ID No: 1 which induce expression of DNA in Tetrahymena are also suitable promoter DNA sequences for use in a chimeric gene of the present invention (infra). The fragments can be prepared by using PCR primers which direct cloning of a smaller portion of the nucleotide sequence of SEQ ID No: 1, and then PCR cloning the desired fragment and isolating the same. The fragment can be inserted into a chimeric gene and the chimeric gene tested to determine whether the fragment is a promoter-effective region. Efficacy of such fragments can be based on a comparison thereof with the full length upstream region of SEQ ID No: 1 (i.e., nt 1-2546).

By virtue of having identified the promoter-effective regions of the 5′ flanking regions of the Tetrahymena MTT1 gene, this promoter can be used to construct a chimeric gene for use in recombinant molecular biology. Thus, a chimeric gene of the present invention will include a first DNA encoding an RNA molecule or a protein or polypeptide (which is to be expressed), a second DNA molecule (which is a Tetrahymena metallothionein gene promoter-effective region of the present invention) operably linked 5′ to the first DNA molecule, and a third DNA molecule comprising a 3′ regulatory region operably linked 3′ to the first DNA molecule.

The first DNA molecule can encode any desired RNA molecule or protein or polypeptide that is to be expressed. In accordance with one aspect of the present invention, the RNA molecule to be expressed can be a non-translatable RNA molecule. Examples of such non-translatable RNA molecules include, without limitation, antisense RNA and inhibitory RNA such as RNA aptamers (Shi et al., “Artificial Genes Expressing RNA Aptamers as Specific Protein Inhibitors in vivo,” Nucleic Acids Symp. Ser. 36:194-196 (1997), which are hereby incorporated by reference). Antisense RNA can be expressed by inserting the DNA coding sequence in reverse orientation relative to the promoter sequence. In accordance with another aspect of the present invention, the RNA molecule to be expressed can be translatable into a protein or polypeptide. The protein or polypeptide can be a homologous (i.e., native) Tetrahymena protein or polypeptide. Such homologous proteins or polypeptides can be expressed at higher levels than normal (following introduction of the chimeric gene into a host cell and induction of the promoter). Alternatively, the protein or polypeptide can be a heterologous protein or polypeptide.

As used herein, the term “heterologous” refers to (i) a DNA segment that has been isolated or derived from one genotype, preferably amplified and/or chemically altered, and later introduced into an organism that may be a different genotype; or (ii) a protein or polypeptide that is not normally expressed (i.e., non-native) within an organism. Heterologous DNA does not generally include DNA of the same genotype, but “heterologous DNA” as used herein also includes DNA of the same genotype from which the amplified, chemically altered, or otherwise manipulated, DNA was first derived. Modification of the heterologous DNA sequence may occur, for example, by treating the DNA with a restriction enzyme to generate a DNA fragment which is capable of being operably linked to a promoter of the present invention. Modification can also occur by techniques such as site-directed mutagenesis or via PCR using primers designed to introduce a particular sequence, such as a restriction site. “Heterologous DNA” also includes DNA that is completely synthetic, semi-synthetic, or biologically derived, such as DNA derived from RNA. “Heterologous DNA” also includes, but is not limited to, genes from other organisms such as those from bacteria, fungi, animals, plants, other protozoans, or viruses; modified genes, portions of genes, chimeric genes, as well as DNA that encodes for amino acids that are chemical precursors or biologics of commercial value, such as polymers or biopolymers (Pool et al., “In Search of the Plastic Potato,” Science 245:1187-1189 (1989), which is hereby incorporated by reference in its entirety). Suitable heterologous DNA is any DNA for which expression in a suitable host cell is desired.

Any of the above-described promoter effective regions can be utilized as the second DNA molecule in constructing the chimeric gene of the present invention.

The third DNA molecule includes an operable 3′ regulatory region. One suitable 3′ regulatory region is the 3′ flanking region of SEQ ID No: 1 (nt 3036 to 3410). Virtually any 3′ regulatory region known to be operable in Tetrahymena or other host cells would suffice for proper expression of the coding sequence of the chimeric gene of the present invention.

The promoter region, the coding region, and the 3′ regulatory region (i.e., the first, second, and third DNA molecules) can be ligated together using well known molecular cloning techniques as described in Sambrook et al., Molecular Cloning: A Laboratory Manual, Second Edition, Cold Spring Harbor Press, NY (1989), which is hereby incorporated by reference in its entirety.

A further aspect of the present invention includes an expression system that includes a suitable expression vector in which is inserted a chimeric gene of the present invention. In preparing the chimeric gene for expression, the various DNA sequences may normally be inserted or substituted into a plasmid. Any convenient plasmid may be employed, which will be characterized by having a suitable replication system, a marker which allows for selection in transformed cells and generally one or more unique, conveniently located restriction sites. Numerous plasmids, referred to as transformation vectors, are available commercially or from other researchers. The selection of a vector will depend on the preferred transformation technique and target species for transformation.

U.S. Pat. No. 4,237,224 issued to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including procaryotic organisms and eukaryotic cells grown in tissue culture.

A further aspect of the present invention includes a host cell which includes a chimeric gene of the present invention. As described more fully hereinafter, the recombinant host cell can be any cell in which the chimeric gene can be replicated (e.g., host bacterium) either with or without expression, as well as host cells in which the chimeric gene can be expressed (e.g., Tetrahymena cells). In the case of recombinant or transgenic Tetrahymena, it is preferable, but not essential, that the chimeric gene is stably inserted into the genome of the recombinant Tetrahymena.

Where matings are made between a knockout Tetrahymena germ line (which either lacks or possesses a disruption in a native Tetrahymena gene, thereby preventing expression of the native Tetrahymena gene) and a recombinant or transgenic Tetrahymena of the present invention, the RNA or protein or polypeptide expressed by the chimeric gene may overcome or diminish the effects caused by non-expression of the native Tetrahymena gene. This is a particularly useful approach for detecting gene-gene interactions.

The chimeric gene can be incorporated into cells using conventional recombinant DNA technology. Generally, this involves inserting the chimeric gene into an expression vector or system to which it is heterologous (i.e., not normally present). As described above, the chimeric gene contains the necessary elements for the transcription and/or translation in host cells of the first DNA molecule.

Once the chimeric gene of the present invention has been prepared, it is ready to be incorporated into a host cell. Recombinant molecules can be introduced into cells via transformation, particularly transduction, conjugation, mobilization, electroporation, or biolistic particle bombardment. The DNA sequences are cloned into the vector using standard cloning procedures in the art, as described by Sambrook et al., Molecular Cloning: A Laboratory Manual, Second Edition, Cold Springs Laboratory, Cold Springs Harbor, N.Y. ( 1989), which is hereby incorporated by reference in its entirety. Suitable host cells include, but are not limited to, bacteria, virus, fungi, mammalian cells, insect, plant, Tetrahymena, and the like. Preferably the host cells are either a bacterial cell or a Tetrahymena (e.g., T. thermophila) cell.

Accordingly, another aspect of the present invention relates to a transgenic Tetrahymena organism that includes a chimeric gene of the present invention. Although it is believed that the chimeric gene of the present invention will operate in any Tetrahymena organism, Tetrahymena thermophila is preferred.

Introduction of the chimeric gene into Tetrahymena can be carried out using any of the above-identified procedures.

One approach to transforming Tetrahymena cells with a chimeric gene of the present invention is particle bombardment (also known as biolistic transformation) (Cassidy-Hanley et al., Genetics 146:135-147 (1997), which is hereby incorporated by reference in its entirety). This can be accomplished in one of several ways. The first involves propelling inert or biologically active particles at cells under conditions effective to penetrate the outer surface of the cell and to be incorporated within the interior thereof. When inert particles are utilized, the vector can be introduced into the cell by coating the particles with the vector containing the chimeric gene. Alternatively, the target cell can be surrounded by the vector so that the vector is carried into the cell by the wake of the particle. Other variations of particle bombardment, now known or hereafter developed, can also be used.

After the transformation procedure, the transformed Tetrahymena can be selected. Typically, selection of transformants is achieved by growing the cultured Tetrahymena in a medium which allows only the transformants to survive. Suitable selection agents include antibiotics which will kill most all non-transformants but allow transformants (which also possess an antibiotic resistance gene) to survive. A number of antibiotic-resistance markers are known in the art and others are continually being identified. Any known antibiotic-resistance marker can be used to transform and select transformed host cells in accordance with the present invention. Similarly, enzymes providing for production of a compound identifiable by color change are useful as selection markers, such as GUS (β-glucuronidase), GFP (green fluorescent protein and its derivatives), or luminescence, such as luciferase.

Another aspect of the present invention relates to a method of expressing an RNA or a protein or polypeptide of interest by providing a chimeric gene of the present invention and transforming a host cell with the chimeric gene under conditions effective to express the RNA or the protein or polypeptide in the host cell as described above. As noted above, the protein or polypeptide which is expressed can be either a homologous or a heterologous protein or polypeptide. With the homologous protein or polypeptide, the promoter of the present invention is capable of inducing high levels of expression of the homologous protein or polypeptide, creating a condition in some instances that may be lethal (i.e., upon induction of expression). Likewise, the same can be determined using expression of heterologous proteins or polypeptides as well as various types of RNA, whereby their inducible expression produces a lethal condition. When the condition is non-lethal, other phenotype changes can be assessed following expression of the RNA or the protein or polypeptide encoded by the chimeric gene. This, too, will allow for an analysis of gene-gene interactions.

In general, expression of the RNA or the protein or polypeptide can be achieved by increasing the concentration of metal ions in the environment of the host cell (i.e., introducing into the media a concentration of metal ions which is sufficient to induce expression of the chimeric gene). One exemplary metal ion is cadmium (Cd²⁺). Other metal ions can be easily identified by introducing metal ion salts into the growth media and assaying for RNA or protein or polypeptide expression, e.g., by Northern or Western blotting techniques. Where expression of the chimeric gene is detected, those metal ions can be considered an inducer-repressor of the promoter of the present invention.

Cessation of chimeric gene expression (assuming non-lethality) can be achieved by withdrawing metal ions from the media using a chelator or by introducing the transformants into fresh media lacking the metal ions. Any suitable chelator can be utilized so long as it is otherwise inert to the transformants.

A further aspect of the present invention relates to an empty expression vector suitable for use in transforming Tetrahymena. The empty vector includes a first DNA molecule comprising one or more restriction enzyme cleavage sites, a second DNA molecule (which is a Tetrahymena metallothionein gene promoter-effective region of the present invention) operably linked 5′ to the first DNA molecule, and a third DNA molecule comprising a 3′ regulatory region operably linked 3′ to the first DNA molecule. Upon insertion of a DNA molecule encoding an RNA or a protein or polypeptide of interest into the first DNA molecule at a cleavage site, the DNA molecule is operably coupled to the second and third DNA molecules. Thus, the empty expression vector can be used to prepare chimeric genes of the present invention for subsequent use in transforming suitable host cells.

### EXAMPLES

The following examples are provided to illustrate embodiments of the present invention but are by no means intended to limit its scope.

Materials & Methods

Strains and Culture Conditions

Wild-type strain CU428 and paclitaxel-sensitive strain CU522 were kindly provided by P. J. Bruns (Cornell University). Knockout heterokaryon strains (GTU1-KO5 and GTU1-KO6) of the GTU1 gene encoding the single γ-tubulin of Tetrahymena were constructed as described by Hai and Gorovsky (Proc. Natl. Acad. Sci. USA 94:1310-1315 (1997), which is hereby incorporated by reference in its entirety). Successful creation of germ-line knockout heterokaryons of the GTU1 gene was demonstrated by the fact that no viable conjugation progeny were obtained when the GTU1 heterokaryons were mated and that progeny could be rescued by transforming with a wild-type GTU1 gene. Strain cTTMG was generated by rescuing the mating GTU1 knockout heterokaryons with a MTT1-driven GTU1 gene. Cells were grown routinely in SPP (1% protease peptone/0.2% glucose/0.1% yeast extract/0.003% EDTA ferric sodium salt) (Gorovsky et al., Methods Cell Biol. IX:311-327 (1975), which is hereby incorporated by reference in its entirety). To starve cells, a log-phase culture was washed and resuspended in 10 mM Tris.HCl (pH 7.5) and incubated for approximately 24 h at 30° C. without shaking. To study induced expression of the MTT1 promoter, different amounts of CdCl₂ were added to growing, starved, or mating cells for the indicated times.

Northern Blot Analysis

RNA was isolated with Trizol (Life Technologies, Grand Island, N.Y.), electrophoresed in 2.2 M formaldehyde-1.2% agarose gels, blotted, and hybridized with [α-³²P]dATP-labeled, randomly primed probes (Ausubel et al., Current Protocols in Molecular Biology (Wiley Interscience, New York) (1988), which is hereby incorporated by reference in its entirety). The probe for rRNA was a 2-kb HindIII fragment from pBS26S encoding the Tetrahymena 26S RNA (Engberg & Nielsen, Nucleic Acids Res. 18:6915-6919 (1990), which is hereby incorporated by reference in its entirety). The GTU1 probe was synthesized from a 1.0-kb StyI-NsiI fragment from pBL-GTU4 (Li, Ph.D. thesis (University of Rochester, Rochester, N.Y.) (1997), which is hereby incorporated by reference in its entirety). The MTT1 probe was a 300-bp PCR product amplified from T. thermophila genomic DNA with coding region primers. Hybridizations were done at 42° C. in 50% formamide, 5X SSC, 1X SPED (0.1% Ficoll/0.1% polyvinylpyrrolidone/0.1% BSA/6 mM SDS/2 mM sodium pyrophosphate/2 mM EDTA), 1% SDS, and 100 μg/ml salmon sperm DNA.

Southern Blot Analysis

To determine the number of copies of the MTT1 gene in T. thermophila, total genomic DNA was isolated from log-phase CU428 cells, digested, electrophoresed, blotted onto Magnagraph nylon membrane (Osmonics Inc., Westborough, Mass.), and hybridized with [α-³²P]dATP-labeled, randomly primed probes using standard protocols (Ausubel et al., Current Protocols in Molecular Biology (Wiley Interscience, New York) (1988), which is hereby incorporated by reference in its entirety).

Western Blotting

Immunoblots of whole-cell protein extracts from Tetrahymena transformed with pBICH3 or pMTT-BICH3 reacted with antibodies to I. multifiliis were prepared as described (Gaertig et al., Nat. Biotechnol. 17:462-465 (1999), which is hereby incorporated by reference in its entirety).

Tetrahymena Transformation

CU428 cells were starved overnight in 10 mM Tris.HCl (pH 7.5) and biolistically transformed with KpnI and SacI digested plasmid pTTMN, p_{Δ}GMN, p_{Δ}MMII, or p_{Δ}GMM using the DuPont Biolistic PDS-1000/He particle delivery system (Bio-Rad) (Cassidy-Hanley et al., Genetics 146:135-147 (1997), which is hereby incorporated by reference in its entirety). After bombardment, cells were resuspended in 50 ml of SPP medium with varying concentrations of CdCl₂, incubated at 30° C. for 3-5 h, and then plated in 96-well microtiter plates in the presence of 120 μg/ml paromomycin sulfate. Plasmid pMTT-BICH3 was linearized with SacI and SalI and used to transform the CU522 strain by biolistic bombardment, as described (Gaertig et al., Nat. Biotechnol. 17:462-465 (1999), which is hereby incorporated by reference in its entirety). Transformants were selected by growth in 20 μM paclitaxel for about 2 weeks. For rescue experiments, plasmid pGTU-E or pTTMG was biolistically transformed into unfed exconjugants from the cross between the GTU1 knockout heterokaryon strains GTUKO5 and GTUKO6, followed by re-feeding with SPP medium with 1.0 μg/ml CdCl₂ and plating.

### Example 1

### Cloning the MTT1 Gene

Based on the amino acid sequence of the T. pyriformis MTT protein (Piccinni et al., Gene 234:51-59 (1999), which is hereby incorporated by reference in its entirety), two degenerate oligonucleotides were synthesized and used to PCR amplify a 300-bp product encoding a fragment of the coding region of the MTT1 gene of T. thermophila. This PCR product was used to probe a genomic Southern blot of T. thermophila DNA digested with TaqI, HindIII, or EcoRI. A single band was observed with each enzyme, indicating that the MTT protein is encoded by a single gene in T. thermophila. Double digestion with HindIII and EcoRI gave a fragment of about 3.2 kb. Size-selected HindIII and EcoRI double-digested genomic DNA was ligated into digested pBluescript KS(+) plasmid (Stratagene) and transformed into E. coli DH5α bacteria. Colony lifts were hybridized with the 300-bp product. A positive colony was subsequently cloned and sequenced, revealing an ORF of 163 codons (including the termination codon) that had very high similarity with MTT from T. pyriformis. This construct, pTTMet, contains ˜2.5 kb of 5′ flanking, 489-bp coding, and ˜375 bp of 3′-flanking region of the MTT1 gene of T. thermophila (see FIG. 1).

The metallothionein (MTT1) gene of T. thermophila (GenBank Accession No. AY061892, which is hereby incorporated by reference in its entirety) encodes a protein (MTT1p) containing 162 amino acids, which is very similar to cadmium-metallothioneins from T. pyriformis and Tetrahymena pigmentosum (Piccinni et al., Eur. J. Protistol. 26:176-181 (1990), Piccinni et al., Gene 234:51-59 (1999), each of which is hereby incorporated by reference in its entirety), except that it contains a duplication corresponding to residues 3-55. The MTT1 gene is present in a single copy gene in T. thermophila, as in T. pyriformis (Piccinni et al., Gene 234:51-59 (1999), which is hereby incorporated by reference in its entirety).

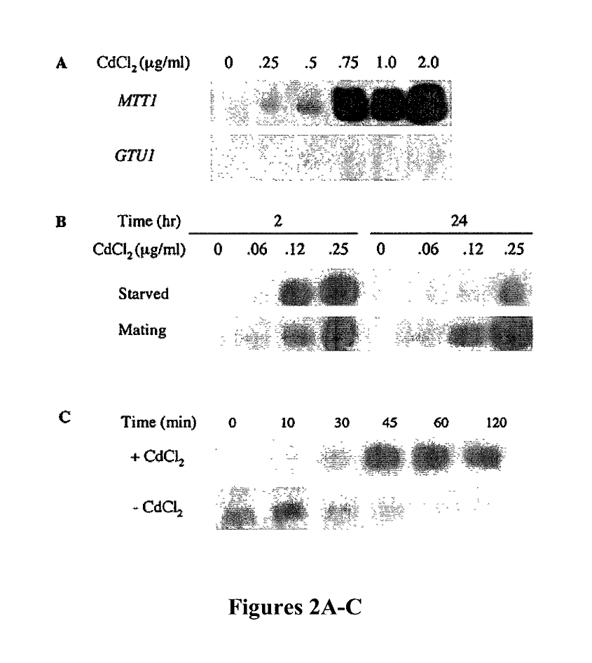

To evaluate the inducibility of the MTT1 promoter in T. thermophila, RNA isolated from log-phase wild-type strain CU428 cells grown overnight in SPP medium containing the indicated concentrations of CdCl₂ was analyzed by Northern blotting. T. thermophila can grow in up to 2.0 μg/ml CdCl₂, and the growth rate in 1.0 μg/ml CdCl₂ is indistinguishable from that in CdCl₂-free medium. The expression of the MTT1 gene is not detectable in the absence of CdCl₂, but can be induced to high levels and can be regulated by the level of CdCl₂ in growing (FIG. 2A) and starved (FIG. 2B) cells. Starved cells were more sensitive to CdCl₂, and MTT1 expression in these cells could be induced at lower concentrations than in growing cells. The mating process was also delayed in the presence of CdCl₂. Three hours after mixing, about 61% of the cells were paired in 0.06 μg/ml CdCl₂, compared with 81% in CdCl₂-free Tris. Induction in mating cells was similar to starved cells (FIG. 2B). Reduction of MTT1 induction in starved cells after 24 h exposure to CdCl₂ (compared with 2 h in FIG. 2B) is reproducible and does not occur in starved-mating cells, but was not investigated further.

To determine the rapidity with which the MTT1 promoter can be activated by CdCl₂, log-phase wild-type cells were incubated in standard culture medium (SPP) with 1.0 μg/ml CdCl₂, and expression of MTT1 was measured after different periods. FIG. 2C indicates that MTT1 expression can be induced very rapidly. MTT1 mRNA was detected within 10 min after adding the CdCl₂ and reached a maximum level at about 45 min. Cessation of MTT1 expression also occurs rapidly after CdCl₂ is removed. In cells that had been growing with 1.0 μg/ml CdCl₂ overnight and were transferred into medium without CdCl₂, a decrease in MTT1 mRNA was evident by 30 min, and by 60 min the MTT1 mRNA could not be detected (FIG. 2C), which suggests that transcription of the MTT1 gene can be turned off very rapidly simply by depleting the medium of cadmium.

### Example 2

### Construction of Transformation Vectors

The reporter construct pTTMN was obtained by replacing the MTT1 coding region with the neo1 coding sequence. Plasmid p4T2-1 is a pBluescript KS (+) derivative containing the neo2 cassette, a chimeric HHF1/neo1/BTU2 gene, with a HindIII site after the neo1 start codon (Gaertig et al., Nucleic Acids Res. 22:5391-5398 (1994), which is hereby incorporated by reference in its entirety). The neo1 coding region was PCR-amplified from p4T2-1, and the fragment was purified after first treating with T4 DNA polymerase followed by HindIII. Using pTTMet and an inverse PCR primer that added a HindIII site after the ATG start codon and a second primer starting ˜120 bp 3′ of the TGA stop codon, the 5′-flanking MTT1-pBluescript vector-3′-flanking MTT1 sequence was amplified. This fragment was treated with T4 DNA polymerase, then digested with HindIII and ligated to the neo1 fragment to create pTTMN.

The pMNBL plasmid, which contains the neo3 cassette, a hybrid MTT1/neo1/BTU2 gene, was constructed as follows. Plasmid pTTMN was digested with KpnI, which cleaves in the multiple cloning site of the pBluescript vector, blunted by T4 DNA polymerase, and digested with HindIII. The 2.5-kb fragment containing the MTT1 5′-flanking sequence was gel purified. p4T2-1 was also digested with KpnI, blunted with T4 DNA polymerase, and digested with HindIII, and the large fragment containing the neo1-coding region-BTU2 3′-flanking-pBluescript vector sequence was isolated. The two fragments were then ligated to construct the pMNBL plasmid. To construct pMNBM, which contains only ˜600 bp of MTT1 5′-flanking sequence upstream of the ATG start codon, the EcoRV-HindIII fragment containing the HHF1 promoter in the p4T2-1 plasmid was replaced by the ˜600-bp AflIII-HindIII fragment from the pTTMN plasmid.

To construct the GTU1 knockout plasmid, p_{Δ}GN, a 0.7-kb fragment of the GTU1 5′-flanking sequence (from a BglII site to the ATG start codon) was PCR-amplified from genomic DNA with use of a forward primer that introduced a KpnI site at its end and a reverse oligo that introduced a NotI site at its end. The PCR fragment was blunted with T4 DNA polymerase, digested with KpnI, and inserted into the 5′ polylinker region (between KpnI and EcoRV) of p4T2-1. A 1.0-kb fragment of the GTU1 3′-flanking sequence was PCR-amplified from Tetrahymena genomic DNA by using a forward primer and a reverse primer that introduced a XhoI and SacI sites at their ends, respectively. This PCR product was blunted with T4 DNA polymerase, digested with SacI, and inserted into the 3′ polylinker region (between SmaI and SacI) of p4T2-1. The plasmid p_{Δ}GMN, which contains 2.5 kb of MTT1 5′-flanking sequence upstream of the ATG start codon, or p_{Δ}GMM, which has only 600 bp of MTT1 5′-flanking sequence, was constructed by subcloning the NotI-XhoI fragment containing the MTT1/neo1/BTU2 gene from either pMNBL or pMNBM into p_{Δ}GN between the NotI and XhoI sites.

To construct p_{Δ}GMMII, which contains the 900 bp of MTT1 5′-flanking sequence directly 5′ of the ATG start codon, p_{Δ}GMN was digested with AccI to release the distal 1.6-kb MTT1 5′-flanking sequence with ˜120 bp of GTU1 3′-flanking sequence. The large fragment obtained from this restriction digestion was self-ligated. In the ngoA gene knockout constructs, p_{Δ}NgoAH4 or p_{Δ}NgoAMT, either the neo2 or neo3 cassette was inserted between the ngoA 5′- and 3′-flanking regions using similar procedures.

To create the gene expression construct pMTT-BICH3, site-directed mutagenesis was used to introduce a BglII site about 490 bp upstream of the ATG start codon within the 5′-flanking region of pBICH3 (Gaertig et al., Nat. Biotechnol. 17:462-465 (1999), which is hereby incorporated by reference in its entirety), which contains the coding region of the IAG48[G1] surface antigen gene of the ciliate Ichthyophthirius multifiliis inserted between the flanking sequences of the BTU1 gene of T. thermophila. A HindIII site exists a few base pairs downstream of the ATG start codon in pBICH3; and a BglII site is very close to the 5′ end of the MTT1 5′-flanking region. The proximal part of the BTU1 promoter was removed by digestion with HindIII and BglII, and replaced by the 2.5-Kb BglII-HindIII fragment of the MTT1 promoter from the pTTMN plasmid.

To create plasmid pTTMG to rescue the progeny of matings of GTU1 knockout heterokaryons, the γ-tubulin coding region was PCR-amplified from pGTU (Li, Ph.D. thesis (University of Rochester, Rochester, N.Y.) (1997), which is hereby incorporated by reference in its entirety), which contains a wild-type version of GTU1. This amplified fragment was ligated to the inverse PCR fragment from pTTMet, containing the pBluescript vector sequence and the 5′- and 3′-flanking sequences of MTT1, as described above. pGTU-E is pGTU with an EcoRI site added by mutagenesis directly 5′ of the TGA and an additional 1.5 kb of 5′-flanking sequence added by inverse PCR.

### Example 3

### Inducible Expression of Reporter Gene neo1 at the MTT1 Locus

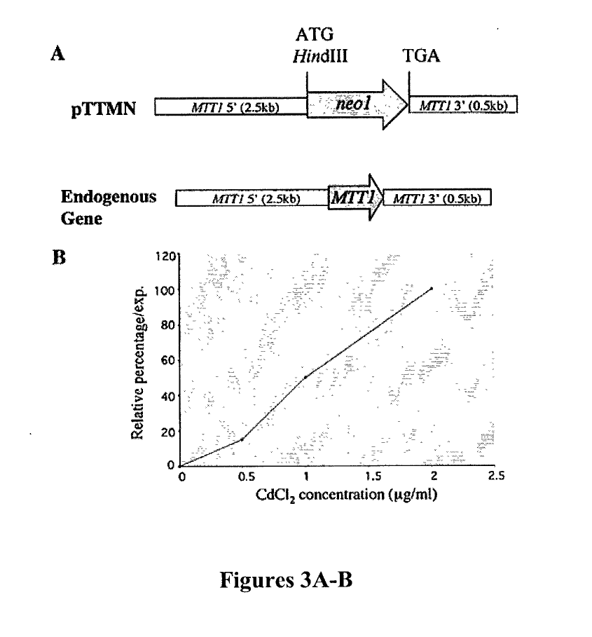

To test whether the MTT1 promoter can induce the expression of other coding regions in response to cadmium, a reporter construct was made by replacing the MTT1 coding region with the neo1 coding region which confers paromomycin (pm) resistance when expressed in Tetrahymena macronuclei (Kahn et al., Proc. Natl. Acad. Sci. USA 90:9295-9299 (1993), which is hereby incorporated by reference in its entirety). Because this reporter gene is flanked by MTT1 noncoding sequences, it integrates into the MTT1 locus by homologous recombination when biolistically transformed into Tetrahymena. After transformation, cells were selected in medium containing paromomycin and increasing amounts of CdCl₂. FIG. 3 indicates that transformation efficiency with this construct is highly dependent on cadmium concentration. No pm-resistant cells were obtained in the absence of cadmium, and the number of transformants was proportional to cadmium concentration. This transformation experiment was repeated three times with similar results, and up to 25,000-30,000 transformants per μg DNA were obtained at 2.0 μg/ml CdCl₂.

To test whether MTT1p is required for survival of Tetrahymena in CdCl₂, transformants were transferred in SPP containing 0.5 μg/ml cadmium with increased concentration of paromomycin for a month. Both genomic PCR and Northern blot analysis showed that the endogenous MTT1 gene was completely replaced. Cells lacking MTT1 can barely grow in 2.0 μg/ml CdCl₂, whereas their growth rate in 1.0 μg/ml CdCl₂ or in cadmium-free medium is indistinguishable and similar to that of wild-type control cells.

### Example 4

### MTT1 Promoter Enables Higher Frequency Gene Disruption in Somatic Cells than the HHF1 Promoter

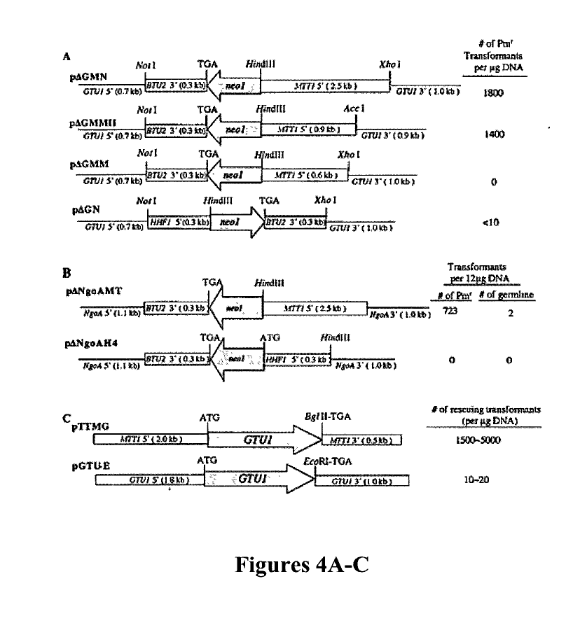

For gene disruption studies in Tetrahymena, the neo2 cassette was developed in which the promoter of the HHF1 gene (encoding histone H4) and the termination region of the BTU2 gene (encoding β-tubulin) were used to express the neo1 coding region (Gaertig et al., Nucleic Acids Res. 22:5391-5398 (1994), which is hereby incorporated by reference in its entirety). To determine whether the MTT1 promoter would allow higher transformation efficiencies if used instead of the HHF1 promoter, the neo3 cassette was created by replacing the HHF1 gene 5′ region of neo2 with the 2.5 kb of MTT1 5′-flanking sequence. To test this new cassette, the knockout plasmid p_{Δ}GMN was constructed in which the neo3 cassette is flanked by the 5′ and 3′ sequences of the GTU1 gene encoding the single γ-tubulin gene of Tetrahymena. Somatic biolistic transformation was performed by using either linearized p_{Δ}GMN or p_{Δ}GN, which contains the neo2 cassette (FIG. 4A). Approximately 1,800 pm-resistant transformants per μg DNA were obtained by using neo3, whereas fewer than 10 transformants per μg DNA were obtained with neo2. Therefore, the new MTT1/neo1/BTU2 cassette enables gene disruption at much higher frequency than the HHF1/neo1/BTU2 cassette.

The amount of MTT1 5′-flanking sequence required for biolistic gene disruption was also examined by using neo3. Plasmid p_{Δ}GMMII, which contains a ˜900-bp 5′-flanking sequence of the MTT1 gene, transformed Tetrahymena at high frequencies, close to those obtained with p_{Δ}GMN containing ˜2.5 kb of flanking sequence. In contrast, p_{Δ}GMM, which contains only a 600-bp flanking sequence, failed to transform Tetrahymena (FIG. 4A). Thus, an important, Cd-responsive promoter element is likely to be in the region 600-900 bp upstream of the MTT1 coding sequence.

### Example 5

### neo3 Cassette Enables Gene Disruption in the Germ Line in Cases Where neo2 Failed

Two genes were unable to be disrupted either in the somatic macronucleus or in the germ-line micronucleus using neo2. These failures likely represent extreme cases of a more general, position-effect phenomenon where the neo2 cassette inserted into different loci produces transformants differing in their resistance to paromomycin by greater than 100-fold.

The ability of the neo3 cassette to disrupt these loci was also tested. ngoA is a gene of unknown function specifically expressed in conjugating cells (Martindale & Bruns, Mol. Cell. Biol. 3:1857-1865 (1983), which is hereby incorporated by reference in its entirety). Although the neo2 knockout construct failed to produce any transformants (FIG. 4B), with the neo3 cassette more than 700 pm-resistant transformants per 12 μg DNA were obtained, two of which were shown by subsequent analyses to be true germ-line knockout transformants. Similarly, for the BLT1 gene encoding a highly divergent β-tubulin present in very low abundance, a disruption construct containing the neo3 cassette produced numerous somatic knockouts and a low number of germ-line knockouts. Thus, in both cases encountered where neo2 failed to produce somatic or germ-line transformants, neo3 was successful.

### Example 6

### MTT1 Promoter Increases Rescue of Knockout Heterokaryons

Rescue of progeny of matings of knockout heterokaryons with in vitro mutagenized genes has proved to be a rapid method for in vivo mutagenesis of essential genes in Tetrahymena (Xia et al., J. Cell Biol. 149:1097-1106 (2000); Ren & Gorovsky, Mol. Cell 7:1329-1335 (2001), each of which is hereby incorporated by reference in its entirety). The frequencies of rescue were measured following mating GTU1 knockout heterokaryon strains by a wild-type GTU1 gene targeted to the GTU1 locus with that of wild-type GTU1 coding region targeted to the MTT1 locus (FIG. 4C). Clearly, rescue by cloning into the MTT1 locus is 2-3 orders of magnitude more efficient than integration into the GTU1 locus itself.

### Example 7

### MTT1 Promoter Enables Analysis of the Hypomorphic or Terminal Phenotype of an Essential Gene by Converting it Into a Conditional Mutant

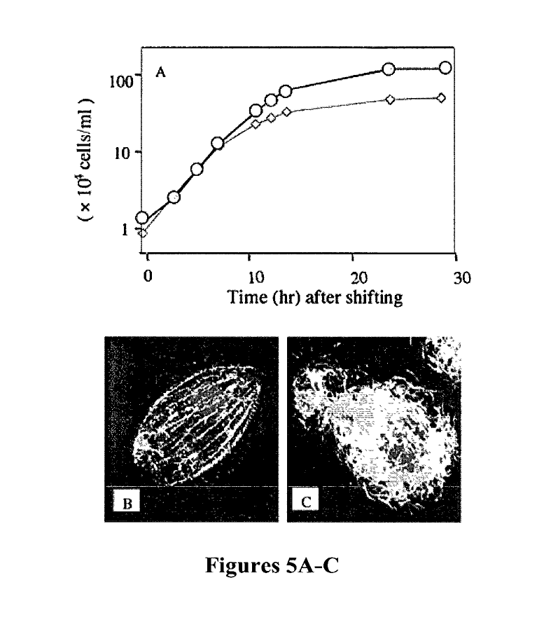

Because knocking out essential genes usually results in dead cells, it is frequently difficult to obtain information on the null phenotype of these genes. This difficulty is a particularly acute problem in Tetrahymena, where many new mutations are the product of reverse genetic analysis of gene knockouts. In other systems, this problem is often addressed by creation of conditional lethal mutations, whose properties can be studied under the nonpermissive condition. To determine whether placing an essential gene under the control of the MTT1 promoter would lead to a conditional, Cd-regulatable mutant, the phenotype of a mutant strain (cTTMG) was examined in which the only expressed GTU1 gene was under MTT1 control. The growth rate of such cells is indistinguishable from that of wild-type cells in the presence of CdCl₂, but they cease growing when resuspended in CdCl₂-free medium (FIG. 5A), and cell shape and the organization of the tubulin cytoskeleton become extremely abnormal (FIG. 5C). This phenotype is similar to the phenotype observed in progeny cells of mating GTU1 knockout heterokaryons, which have only disrupted copies of GTU1. Although the detailed phenotype of these cells in the absence of CdCl₂ will be presented elsewhere, it is clear from these studies that the wild-type GTU1 gene exhibits a conditional phenotype in the absence of CdCl₂ that is likely caused by γ-tubulin depletion.

### Example 8

### Overexpression of an Ich Surface Antigen Gene Driven by the MTT1 Promoter

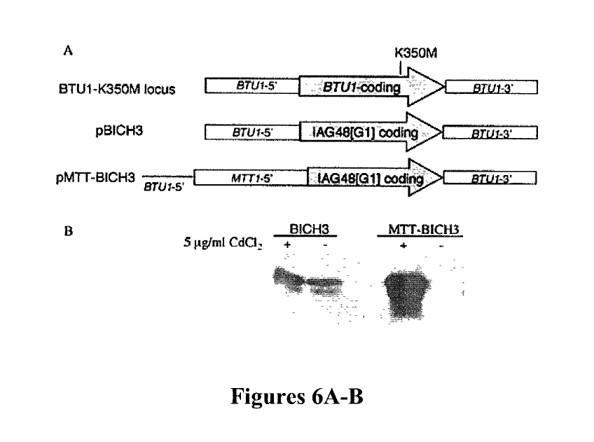

The BTU1 gene is one of two co-expressed genes encoding the major β-tubulin of T. thermophila (Gaertig et al., Cell Motil. Cytoskeleton 25:243-253 (1993); Gu et al., Mol. Cell. Biol. 15:5173-5179 (1995), each of which is hereby incorporated by reference in its entirety). BTU1 mRNA is highly abundant and β-tubulin makes up about 2-3% of the total Tetrahymena cell protein (Calzone, Ph.D. thesis (University of Rochester, Rochester, N.Y.) (1982)). It was shown previously that this highly active promoter could drive high-level expression of the IAG48[G1] surface antigen gene of the parasite ciliate I. multifiliis inserted into the Tetrahymena BTU1 locus in place of the BTU1 coding region (Gaertig et al., Nat. Biotechnol. 17:462-465 (1999), which is hereby incorporated by reference in its entirety). To determine whether the MTT1 promoter would be useful for overexpression of heterologous genes in Tetrahymena, the expression levels of the IAG48[G1] surface antigen gene driven by the MTT1 promoter was compared with that driven by the BTU1 promoter. In the pMTT-BICH3 construct, the 2.5-kb MTT1 5′-flanking region was inserted upstream of the coding region of the IAG48[G1] surface antigen gene in the previously described pBICH3 plasmid which also contains BTU1 3′- and 5′-flanking sequences (FIG. 6A). Both constructs were then (separately) transformed biolistically into the BTU1 locus of strain CU522, which contains a dominant, paclitaxel-sensitive BTU1 gene (Gaertig et al., Nat. Biotechnol. 17:462-465 (1999); Gaertig et al., Proc. Natl. Acad. Sci. USA 91:4549-4553 (1994), each of which is hereby incorporated by reference in its entirety). Replacement of the paclitaxel-sensitive gene by the insertion of either plasmid results in paclitaxel-resistant cells, which are easily selected. Expression of Ich surface antigen genes in both transformed strains was measured by Western blotting with and without treating the cells with CdCl₂ (FIG. 6B). The expression level of the Ich surface antigen driven by the MTT1 promoter was much higher than that driven by the BTU1 promoter. After optimization for CdCl₂ concentration (5 μg/ml) and time of treatment (9 h), quantitative analyses of similar gels indicated that the expression of the Ich surface antigen driven by the MTT1 promoter was 18-30 times greater than from the BTU1 promoter and constituted up to ˜1% of the total cell protein. Similarly, the γ-tubulin can be overexpressed in cells in which the GTU1 gene is regulated by the MTT1 promoter. Clearly, the MTT1 promoter is now the promoter of choice for overexpression of genes in Tetrahymena.

### Discussion of Examples 1-8

The T. thermophila metallothionein gene (MTT1) promoter is highly regulatable and can be used to increase the efficiency of most of the commonly used types of DNA-mediated transformation in this organism. The MTT1 promoter can be expressed in a graded fashion in proportion to CdCl₂ concentration in growing, starved, and conjugating cells. This promoter can be turned on and off rapidly, suggesting it can be used to study the site and kinetics of incorporation and turnover of MTT1-regulated tagged genes by treating cells briefly with CdCl₂. The MTT1 promoter is able to highly overexpress both homologous and heterologous genes and the fact that the MTT1 coding region is not essential indicates that Tetrahymena should be useful as an inexpensive, easy-to-grow, eukaryotic expression system for foreign genes.

Many lines of evidence suggest that the MTT1 promoter is tightly regulated. In the absence of CdCl₂, MTT1 expression was not detected in growing, starved, and mating cells, and no transformants were obtained when the neo1 coding region was used to disrupt the MTT1 gene. In addition, the growth and microtubule organization of cells whose GTU1 gene was regulated by the MTT1 promoter was markedly altered when CdCl₂ was removed from the medium. These observations indicate that the MTT1 promoter enables fine control of gene expression-induction over a wide range in the presence of inducer, and tight repression in its absence. However, although depletion of cadmium resulted in the cessation of growth in cells containing MTT1-GTU1 chimeric genes, cells left in growth medium in the absence of cadmium eventually recovered normal morphology and resumed growth. Very low expression of GTUp was detected in these recovered cells on Western blots. Leaky expression has also been observed after the depletion of cadmium when MTT1 promoter-driven genes are inserted into the BTU1 locus. The GTU1 gene is only weakly detected on Northern blots in wild-type cells, suggesting that it provides an especially sensitive test of leaky expression. On the other hand, cells containing a MTT1-BTU1 chimeric gene as their only major β-tubulin gene do not resume growth, even when maintained for a week after being resuspended in cadmium-free medium. These observations suggest that the tightness and consequences of MTT1 promoter silencing in the absence of cadmium may show some locus-specific effects. In summary, the T. thermophila metallothionein gene promoter is a robust, inducible promoter that greatly enhances molecular genetic analyses in Tetrahymena.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. An isolated DNA molecule comprising a promoter-effective region of a Tetrahymena metallothionein gene.

2. The isolated DNA molecule according to claim 1 wherein the DNA molecule comprises greater than about 600 bp upstream of the Tetrahymena metallothionein gene start codon.

3. The isolated DNA molecule according to claim 1 wherein the DNA molecule comprises at least about 900 bp upstream of the Tetrahymena metallothionein gene start codon.

4. The isolated DNA molecule according to claim 1 wherein the DNA molecule comprises at least about 1.6 kb upstream of the Tetrahymena metallothionein gene start codon.

5. The isolated DNA molecule according to claim 1 wherein the DNA molecule comprises at least about 2.5 kb upstream of the Tetrahymena metallothionein gene start codon.

6. The isolated DNA molecule according to claim 5 wherein the DNA molecule is a BglII-HindIII fragment of plasmid pTTMN.

7. The isolated DNA molecule according to claim 1 wherein the DNA molecule comprises bases 1-2546 of SEQ ID No: 1 or promoter-effective fragments thereof.

8. A chimeric gene comprising:
a first DNA encoding an mRNA molecule or a protein or polypeptide;
a second DNA molecule according to claim 1 operably linked 5′ to the first DNA molecule; and
a third DNA molecule comprising a 3′ regulatory region operably linked 3′ to the first DNA molecule.

9. The chimeric gene e according to claim 8 wherein the first DNA molecule comprises bases 1-2546 of SEQ ID No: 1 or promoter-effective fragments thereof.

10. An expression vector comprising a chimeric gene according to claim 8.

11. A host cell comprising a chimeric gene according to claim 8.

12. The host cell according to claim 11, wherein the host cell is a Tetrahymena cell.

13. A transgenic Tetrahymena organism comprising a chimeric gene according to claim 8.

14. The transgenic Tetrahymena organism according to claim 13 wherein the Tetrahymena organism is Tetrahymena thermophila.

15. The transgenic Tetrahymena organism according to claim 13 wherein the Tetrahymena organism either lacks or possesses a disruption in a native Tetrahymena gene, thereby preventing expression of the native Tetrahymena gene.

16. The transgenic Tetrahymena organism according to claim 13 wherein the RNA or protein or polypeptide expressed by the chimeric gene overcomes or diminishes the effects caused by non-expression of the native Tetrahymena gene.

17. A method of expressing an RNA or a protein or polypeptide of interest comprising:
providing a chimeric gene according to claim 8 and
transforming a host cell with the chimeric gene under conditions effective to express the RNA or the protein or polypeptide in the host cell.

18. The method according to claim 17 wherein the host cell is a Tetrahymena cell.

19. The method according to claim 17 wherein the RNA is expressed.

20. The method according to claim 19 wherein the RNA is antisense RNA or an RNA aptamer.

21. The method according to claim 17 where the protein or polypeptide is expressed.

22. The method according to claim 21 wherein the expressed protein or polypeptide is a homologous Tetrahymena protein or polypeptide that is expressed at levels higher than normal.

23. The method according to claim 22 wherein overexpression of the homologous Tetrahymena protein or polypeptide produces a lethal condition.

24. The method according to claim 21 wherein the expressed protein or polypeptide is a heterologous protein or polypeptide.

25. The method according to claim 24 wherein expression of the heterologous protein or polypeptide produces a lethal condition.

26. The method according to claim 17 wherein the conditions effective for expression of the RNA or the protein or polypeptide comprise increasing the concentration of metal ions in the environment of the host cell.

27. The method according to claim 26 wherein the metal ions are cadmium ions.

28. An empty expression vector comprising:
a first DNA molecule comprising one or more restriction enzyme cleavage sites;
a second DNA molecule according to claim 1 coupled 5′ of the first DNA molecule; and
a third DNA molecule comprising a 3′ regulatory region operably linked 3′ of the first DNA molecule.

29. The empty expression vector according to claim 28 wherein the second DNA molecule comprises bases 1-2546 of SEQ ID No: 1.
